(19) United States
(12) Patent Application Publication
(10) Pub. No.: US 2008/0213856 A1
(43) Pub. Date: Sep. 4, 2008

(54) Method and Device for Isolating Micro-Organisms

(76) Inventors: Nicolas Bara, Paris (FR); Thierry Bernardi, Perignat-les-Sarlieve (FR)

Correspondence Address: IP GROUP OF DLA PIPER US LLP, ONE LIBERTY PLACE, 1650 MARKET ST, SUITE 4900, PHILADELPHIA, PA, 19103, US

(21) Appl. No.: 11/886,082

(22) PCT Filed: Mar. 15, 2006
(86) PCT No.: PCT/FR2006/000578

§ 371 Date: Dec. 12, 2007

(30) Foreign Application Priority Data
Mar. 15, 2005 (FR) 0502553

(51) Int. Cl. C12N 13/00; C12Q 1/24

(52) U.S. Cl. 435/173.9; 435/261

(57) ABSTRACT

A process for isolating at least one micro-organism from a medium including a) introducing a selected quantity of magnetic or magnetizable particles into a sample of the medium; b) incubating the particles and the medium for a time sufficient for the micro-organisms to develop and adhere to surfaces of the particles; c) separating the particles from the medium; d) spreading the particles on a support compatible with development of the micro-organisms; and e) incubating the particles on the support for a time sufficient for the development of colonies corresponding to the isolated micro-organism.

### RELATED APPLICATION

This is a §371 of International Application No. PCT/FR2006/000578, with an international filing date of Mar. 15, 2006 (WO 2006/097631 A1, published Sep. 21, 2006), which is based on French Patent Application No. 05/02553, filed Mar. 15, 2005.

### TECHNICAL FIELD

This disclosure relates to the isolation of micro-organisms.

### BACKGROUND

Many micro-organisms develop by synthesizing a biofilm. Besides bacteria, fungi, algae, and protozoa are also organized in biofilms. Biofilms are thus found in numerous fields where they involve health risks and may cause relatively important damages.

When a biofilm develops, bacteria first adhere to a support and then colonize the support. When multiplying, the bacteria rapidly form a film constituted of cell bodies layers which secrete a matrix of exopolysaccharides which protect them against the environmental stress (COSTERTON and al., Science, vol. 284 (5418), p: 1318-22, 1999). The kinetics of the form ation of a biofilm can be subdivided into 5 steps as shown in FIG. 1:
surface conditioning: the organic or mineral molecules existing in the liquid phase will adsorb on the surface and will form a “conditioning film.”
the adherence or reversible adhesion: the existing micro-organisms get closer to the surfaces by gravimetry, Brownian movements or chemiotaxis, if they have flagella. During this first fixing step, wherein only purely physical phenomena and weak physido-chemical interactions are executed, the micro-organisms can still be easily disconnected.
the adhesion: this step is slower and requires interactions of higher energy as well as the microbial metabolism and the cell appendices of the micro-organism (flagella, pili, . . . ). Adhesion is an active and specific phenomenon. The first colonizing micro-organisms will irreversibly cling to the surface, more particularly through the synthesis of exopolysaccharides (EPS). This process is relatively slow and depends on the environmental factors and the existing micro-organisms.
the maturation of the biofilm (development and colonisation of the surface): when they have adhered to a surface, the bacteria multiply and gather together to form micro-colonies surrounded by polymers. Such polymer matrix (or glycocalix) will act as a “cement” and reinforce the association of bacteria with one another and with the surface, to finally form a biofilm and reach a state of equilibrium. The biofilm generally develops into a three-dimension structure, which constitutes a confinement place. Such micro-environment will be the head quarters of numerous physiological and molecular modifications with respect to the plankton growth mode. If the conditions are favourablefavorable, the thus formed biofilm will fill the whole surface that is available. The maturation of the biofilm is generally correlated to the production of EPS, even though some species of micro-organisms which synthesize few or no polymers can also adhere and form biofilms on surfaces.
Disconnection: Biofilms are structures in perpetual dynamic equilibrium which evolve according to the support, the micro-organisms and the environment. Such evolution can result in cells or aggregates disconnection.

### SUMMARY

We provide a process for isolating at least one micro-organism from a medium including a) introducing a selected quantity of magnetic or magnetizable particles into a sample of the medium; b) incubating the particles and the medium for a time sufficient for the micro-organisms to develop and adhere to surfaces of the particles; c) separating the particles from the medium; d) spreading the particles on a support compatible with development of the micro-organisms; and e) incubating the particles on the support for a time sufficient for the development of colonies corresponding to the isolated micro-organism.

### BRIEF DESCRIPTION OF THE DRAWINGS

Other advantages will appear in the appended Figures, in which:

FIG. 1 shows a comparison of conventional methods for sampling micro-organisms (A) and the method (B);

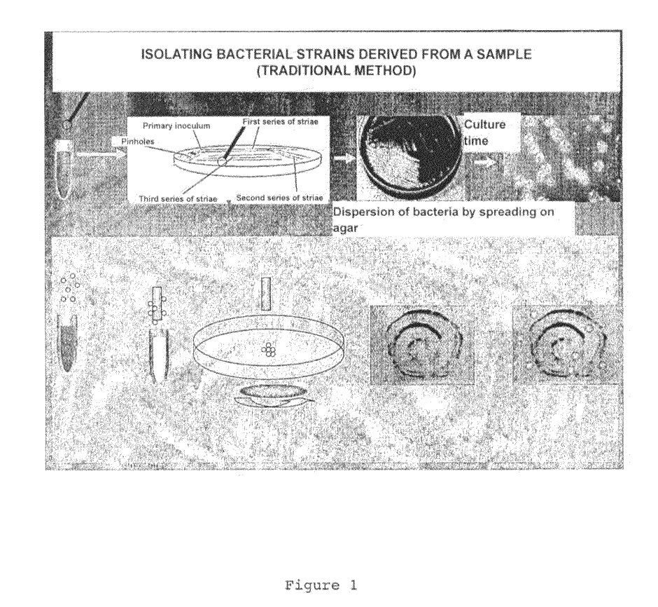

FIG. 2 shows the steps of sampling and washing the particles;

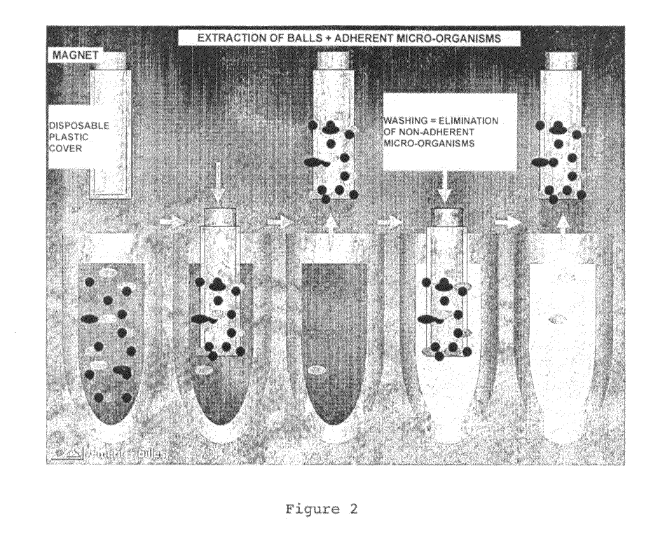

FIG. 3 shows the deposition of particles onto the surface of a Petri box; and

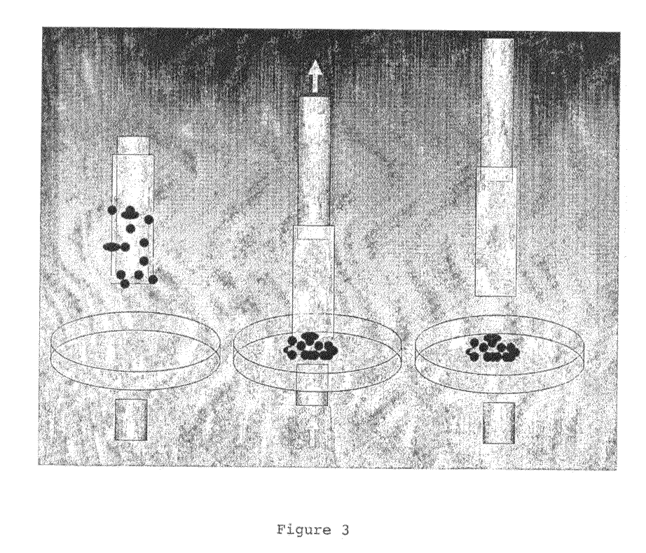

FIG. 4 shows the dispersion of the particles on the surface of the Petri box, using a rotating magnet.

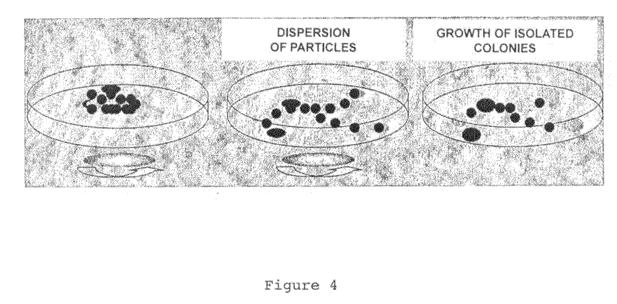

### DETAILED DESCRIPTION

According to our methods, the adhesion and fixation property of micro-organisms is used to isolate them.

Our methods include adding into a more or less liquid raw sample of a medium, the micro-organisms contamination of which is to be studied, magnetic or magnetizable particles and more particularly balls, leave the balls in contact with the medium for a time sufficient for the micro-organisms to adhere to the particles surfaces, isolate the particles using all appropriate means, particularly a magnet and spread the particles on an appropriate solid culture medium to obtain a culture of the micro-organisms trapped in the particles.

Thus, we provide a process for isolating at least a micro-organism from the medium which contains them, comprising the following steps:
a) introducing a given quantity of magnetic or magnetizable particles into a sample of the medium;
b) incubating the particles and the medium for a sufficient time for the micro-organisms to adhere to the surface of the particles;
c) separating the particles from the medium;
d) spreading the particles on a support compatible with the development of the micro-organisms;
e) incubating the particles on a support for a sufficient time for the development of colonies corresponding to the isolated micro-organism.

The process may comprise a prior step of pre-culture of the sample from the medium containing the micro-organism to be isolated. Therefore, the sample is heated to a temperature compatible with the viability of the micro-organisms. It is known that, in addition to the organisms which live at conventional temperatures (20 to 50 degrees Celsius), some micro-organisms live in extreme conditions, as regards temperature, gas partial pressure (Oxygen, nitrogen, carbon dioxide, . . . ), salinity, pH (acid, basic), redox and or in aerobic or anaerobic conditions. Most currently, culture temperatures may be between 20 and 50 degrees Celsius, preferably between 30 and 40 degrees Celsius. Such a step of pre-culture, which enriches the culture medium with micro-organisms, can be carried out in an extensively variable time, depending on the micro-organisms, which can extend between 20 minutes and 7 to 10 days, preferably between 1 hour and 48 hours, while possibly being stirred.

The process may also include an additional step between steps c) and d) of the process which includes optional immersion of the balls obtained in step c) into an advantageously aqueous washing solution, which makes it possible to eliminate the non adherent micro-organisms (which are present in the ball saturation (“imbibition”) liquid). Such step makes it possible to select the most adherent micro-organisms whose adhesion is the most irreversible. Such step may also be the opportunity of applying rapid or extended treatments with a view to testing the adhesion property of micro-organisms (preventive or curative treatments in the washing solution).

Those skilled in the art know how to determine without any difficulty the quantity of balls to be introduced into the medium.

The incubation of step b) carries on for a time which can last from a few seconds to a few hours, preferably between 15 seconds and 45 minutes, depending on the micro-organisms.

After the incubation time, the micro-organisms have had the possibility of adhering to the particles (balls).

Separation of the particles and the medium in step c) can be carried out by any method known to one skilled in the art. For example, such particles can be sampled by centrifugation and elimination of the culture medium, or further, and preferably by using a system generating a magnetic or electric field capable of attracting the particles, particularly a magnet. According to this particularly preferred aspect, the particles are sampled using a magnet which is advantageously dipped into the sample.

The system generating a magnetic or electric field capable of attracting the particles, particularly a magnet, may be protected, by any system, particularly by a removable coating or a cover, made of any material, for example, plastics, which does not interfere with magnetic or electric waves. More advantageously still, the cover is disposable after use. Such a magnet could then be used again.

The method may also include an additional step of washing the system generating a magnetic or electric field to eliminate the non-adherent micro-organisms which are present in the wetting liquid, or the micro-organisms which do not adhere much. During this additional step, the system is dipped into a washing solution which may be a sterile culture medium, for instance. Those skilled in the art understand that, in principle, such a step only lasts for a few seconds, at most a few minutes, which is the time required for eliminating the micro-organisms which did not adhere to the system surface.

Spreading the particles on a support compatible with the development of the micro-organisms can be carried out by deposition of the particles onto the surface of a micro-organism culture device, for example, a Petri dish containing an appropriate culture medium for the development of the micro-organisms.

Deposition can be carried out by taking the magnet out of the plastic cover while placing the plastic cover closer to the micro-organism culture device surface.

The balls may be deposited using another magnet placed under the culture device surface.

Any known system such as a manual spreader, for example, can be used for spreading and dispersing the balls. A rotating magnet may be used and placed under the micro-organism culture device surface.

The ball dispersion can also be obtained by a liquid vortex generated by rotation of the micro-organism culture device.

When the particles (balls) are dispersed, the culture device is placed into an incubator for a time sufficient for the micro-organisms to develop on the device surface. Then, the incubation time and the temperature to the micro-organism to be isolated can be adapted. This time can be between a few hours and several days, preferably between 4 days and 48 days. The incubation temperature can be between 30 and 40 degrees Celsius.

1-12. (canceled)

13. A process for isolating at least one micro-organism from a medium comprising:
a) introducing a selected quantity of magnetic or magnetizable particles into a sample of the medium;
b) incubating the particles and the medium for a time sufficient for the micro-organisms to develop and adhere to surfaces of the particles;
c) separating the particles from the medium;
d) spreading the particles on a support compatible with development of the micro-organisms; and
e) incubating the particles on the support for a time sufficient for the development of colonies corresponding to the isolated micro-organism.

14. The process according to claim 13, further comprising pre-culturing the sample from the medium containing the micro-organism to be isolated, optionally while the medium is stirred.

15. The process according to claim 14, wherein the sample is heated to a temperature compatible with viability of the micro-organisms.

16. The process according to claim 14, wherein the temperature is 20 to 50 degrees Celsius.

17. The process according to claim 14, wherein the temperature is 30 to 40 degrees Celsius.

18. The process according to claim 14, wherein pre-culturing has a duration between 20 minutes and 10 days.

19. The process according to claim 14, wherein the duration is 1 hour to 48 hours.

20. The process according to claim 13, further comprising, between steps c) and d), immersing the particles obtained in step c) into an advantageously aqueous washing solution.

21. The process according to claim 13, wherein incubating is carried out for a few seconds to a few hours, depending on the micro-organisms.

22. The process according to claim 13, wherein incubating is carried out for 15 seconds to 45 minutes.

23. The process according to claim 13, wherein separating the particles and the medium is carried out by centrifugation and elimination of the culture medium, or by using a system generating a magnetic or electric field capable of attracting the particles.

24. The process according to claim 13, wherein separating the particles and the medium is carried out using a magnet.

25. The process according to claim 24, wherein the magnet is dipped into the sample.

26. The process according to claim 13, wherein the system generating a magnetic or an electric field capable of attracting the particles is protected by a removable coating or a cover.

27. The process according to claim 26, wherein the removable coating or cover is disposable.

28. The process according to claim 13, further comprising washing the system generating a magnetic or electric field.
